(19) United States
(12) Patent Application Publication
(10) Pub. No.: US 2015/0335761 A1
(43) Pub. Date: Nov. 26, 2015

(54) COMPOSITIONS AND METHODS FOR CONTRACEPTION

(71) Applicant: Raymond Firestone, New York, NY (US)

(72) Inventor: Raymond Firestone, New York, NY (US)

(21) Appl. No.: 14/377,518

(22) PCT Filed: Jan. 1, 2013
(86) PCT No.: PCT/US2013/024268

§ 371 Date: Aug. 8, 2014

Related U.S. Application Data
(60) Provisional application No. 61/599,544, filed on Feb. 16, 2012.

(51) Int. Cl. A61K 47/48; C07K 5/065

(52) U.S. Cl. CPC A61K 47/48246; C07K 5/06078

(57) ABSTRACT

The invention generally relates to novel compounds, compositions, devices and methods for contraception. More particularly, the invention relates to cytotoxic agents and di-peptide conjugated contraceptive agents, methods for their preparation, pharmaceutical compositions, devices and uses thereof, especially in the prevention of pregnancy.

### PRIORITY CLAIMS AND RELATED PATENT APPLICATIONS

This application is the national phase of PCT/US13/24268, filed Feb. 1, 2013, which claims the benefit of priority from U.S. Provisional Application Ser. No. 61/599,544, filed on Feb. 16, 2012, the entire content of which is incorporated herein by reference in its entirety.

### TECHNICAL FIELDS OF THE INVENTION

The invention generally relates to novel compounds, compositions, devices and methods for contraception. More particularly, the invention relates to cytotoxic agents and di-peptide conjugated contraceptive agents, methods for their preparation, pharmaceutical compositions, devices and uses thereof, especially in the prevention of pregnancy.

### BACKGROUND OF THE INVENTION

Typical birth control methods include contraception and contragestion. The former includes barrier methods and hormonal contraception, while the later includes intrauterine devices and the so-called morning after pills. Contraceptives are designed to prevent fertilization. For example, barrier contraceptives (e.g., condoms, female condoms, cervical caps, and diaphragms) are devices that physically prevent sperm from entering the uterus. Hormonal contraceptives (injectable and oral) inhibit female ovulation or fertilization. Contragestive agents or devices act after fertilization of the egg and before implantation of the embryo into the uterine lining through interruption or prevention of implantation of the blastocyst.

A few birth control methods may be effective if used consistently and correctly, although the most effective methods are those that do not depend upon regular user action. Condoms and cervical barriers such as the diaphragm have typical use first-year failure rates of about 14 and 20 percent, respectively. Some intrauterine devices (IUDs) have been associated with health risks. Side effects may be limited to general feelings of discomfort, nausea or irregular menstrual cycles to some users, while to others it may cause more serious complications. Hormonal contraceptives should not be used by females who suffer from liver cirrhosis, hypertension, diabetes mellitus, and tumors (cancers in general and more specific of cervix or breast tissues), nor should they be prescribed to people who have clotting defects of any kind or genetic predisposition for rheumatoid arthritis. There has also been report that long-term taking of certain hormonal contraceptives may lead to a deficiency of folic acid, which could lead to cardiovascular diseases.

Thus, there is a continued unmet need for novel and effective birth control agents, methods and/or devices that are effective and are not accompanied by harmful side effects commonly associated with existing agents or devices.

### DEFINITIONS

Definitions of specific functional groups and chemical terms are described in more detail below. General principles of organic chemistry, as well as specific functional moieties and reactivity, are described in “Organic Chemistry”, Thomas Sorrell, University Science Books, Sausalito: 2006.

Certain compounds of the present invention may exist in particular geometric or stereoisomeric forms. The present invention contemplates all such compounds, including cis- and trans-isomers, R- and S-enantiomers, diastereomers, (D)-isomers, (L)-isomers, the racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention.

Isomeric mixtures containing any of a variety of isomer ratios may be utilized in accordance with the present invention. For example, where only two isomers are combined, mixtures containing 50:50, 60:40, 70:30, 80:20, 90:10, 95:5, 96:4, 97:3, 98:2, 99:1, or 100:0 isomer ratios are contemplated by the present invention. Those of ordinary skill in the art will readily appreciate that analogous ratios are contemplated for more complex isomer mixtures.

If, for instance, a particular enantiomer of a compound of the present invention is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic methods well known in the art, and subsequent recovery of the pure enantiomers.

Given the benefit of this disclosure, one of ordinary skill in the art will appreciate that synthetic methods, as described herein, may utilize a variety of protecting groups. By the term “protecting group”, as used herein, it is meant that a particular functional moiety, e.g., O, S, or N, is temporarily blocked so that a reaction can be carried out selectively at another reactive site in a multifunctional compound. In preferred embodiments, a protecting group reacts selectively in good yield to give a protected substrate that is stable to the projected reactions; the protecting group should be selectively removable in good yield by preferably readily available, non-toxic reagents that do not attack the other functional groups; the protecting group forms an easily separable derivative (more preferably without the generation of new stereogenic centers); and the protecting group has a minimum of additional functionality to avoid further sites of reaction. Oxygen, sulfur, nitrogen, and carbon protecting groups may be utilized. Examples of a variety of protecting groups can be found in Protective Groups in Organic Synthesis, Third Ed. Greene, T. W. and Wuts, P. G., Eds., John Wiley & Sons, New York: 1999.

It will be appreciated that the compounds, as described herein, may be substituted with any number of substituents or functional moieties.

As used herein, the term “pharmaceutically acceptable salt” refers to either a pharmaceutical acceptable acid addition salt or a pharmaceutically acceptable base addition salt of a currently disclosed compound that may be administered without any resultant substantial undesirable biological effect(s) or any resultant deleterious interaction(s) with any other component of a pharmaceutical composition in which it may be contained.

As used herein, the term “pharmaceutically acceptable ester,” refers to esters that hydrolyze in vivo and include those that break down readily in the human body to leave the parent compound or a salt thereof. Suitable ester groups include, for example, those derived from pharmaceutically acceptable aliphatic carboxylic acids, particularly alkanoic, alkenoic, cycloalkanoic and alkanedioic acids, in which each alkyl or alkenyl moiety, for example, having not more than 22 carbon atoms. Examples of particular esters include formates, acetates, propionates, butyrates, acrylates and ethylsuccinates.

As used herein, (Cₓ-C_{y}) refers in general to groups that have from x to y (inclusive) carbon atoms. Therefore, for example, C₁-C₆ refers to groups that have 1, 2, 3, 4, 5, or 6 carbon atoms, which encompass C₁-C₂, C₁-C₃, C₁-C₄, C₁-C₅, C₂-C₄, C₂-C₅, C₂-C₆, and all like combinations. (C₁-C₂₀) and the likes similarly encompass the various combinations between 1 and 20 (inclusive) carbon atoms, such as (C₁-C₆), (C₁-C₁₂) and (C₃-C₁₂).

As used herein, the term “(Cₓ-C_{y})alkyl” refers to a saturated linear or branched free radical consisting essentially of x to y carbon atoms, wherein x is an integer from 1 to about 10 and y is an integer from about 2 to about 20. Exemplary (Cₓ-C_{y})alkyl groups include “(C₁-C₂₀)alkyl,” which refers to a saturated linear or branched free radical consisting essentially of 1 to 20 carbon atoms and a corresponding number of hydrogen atoms. Exemplary (C₁-C₂₀)alkyl groups include methyl, ethyl, n-propyl, isopropyl, n-butyl, isobutyl, dodecanyl, etc. Of course, other (C₁-C₂₀)alkyl groups will be readily apparent to those of skill in the art given the benefit of the present disclosure.

As used herein, the term, “(Cₓ-C_{y})alkoxy” refers to a straight or branched chain alkyl group consisting essentially of from x to y carbon atoms that is attached to the main structure via an oxygen atom, wherein x is an integer from 1 to about 10 and y is an integer from about 2 to about 20. For example, “(C₁-C₂₀)alkoxy” refers to a straight or branched chain alkyl group having 1-20 carbon atoms that is attached to the main structure via an oxygen atom, thus having the general formula alkyl-O—, such as, for example, methoxy, ethoxy, propoxy, isopropoxy, n-butoxy, sec-butoxy, tert-butoxy, pentoxy, 2-pentyl, isopentoxy, neopentoxy, hexoxy, 2-hexoxy, 3-hexoxy, and 3-methylpentoxy.

As used herein, the term “halogen” refers to fluorine (F), chlorine (Cl), bromine (Br), or iodine (I).

In general, the “effective amount” of an active agent refers to an amount sufficient to elicit the desired biological response. As will be appreciated by those of ordinary skill in this art, the effective amount of a compound of the invention may vary depending on such factors as the desired biological endpoint, the pharmacokinetics of the compound, the disease being treated, the mode of administration, and the patient.

### SUMMARY OF THE INVENTION

The invention is based, in part, on the discovery of di-peptide conjugated contragestive and/or contraceptive agents, compositions thereof, and devices and methods of use thereof related thereto for achieving birth control with high effectiveness and minimal side effects. The invention addresses the shortcomings and inadequacies of the existing birth control agents and devices, particularly in substantially minimizing side effects.

In one aspect, the invention generally relates to a unit dosage formulation useful as a contragestive and/or contraceptive agent, comprising a compound of Formula I

T-A₁-A₂-L-(X)ₙ (I)

or a pharmaceutically acceptable salt or ester thereof, in an amount effective in contragestion or contraception, and a pharmaceutically acceptable carrier, wherein

T is a terminal group;

each of A₁ and A₂ is independently an amino-acid group;

L is a single bond,

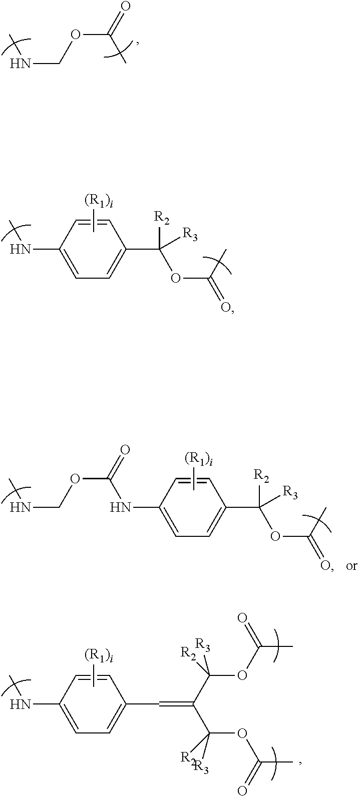

wherein each R₁, R₂ and R₃ is independently a hydrogen, a C₁-C₆ alkyl group, a halogen, a C₁-C₆ alkoxy group; and
X is a group comprising a cytotoxic moiety, and n is 1 or 2.

In another aspect, the invention generally relates to a method for birth control comprising administering to the uterus of a subject in need thereof a contragestive and/or contraceptive effective amount of a unit dosage comprising a compound of Formula I

T-A₁-A₂-L-(X)ₙ (I)

or a pharmaceutically acceptable salt or ester thereof, and a pharmaceutically acceptable carrier, wherein

T is a terminal group;

each of A₁ and A₂ is independently an amino-acid group;

L is a single bond,

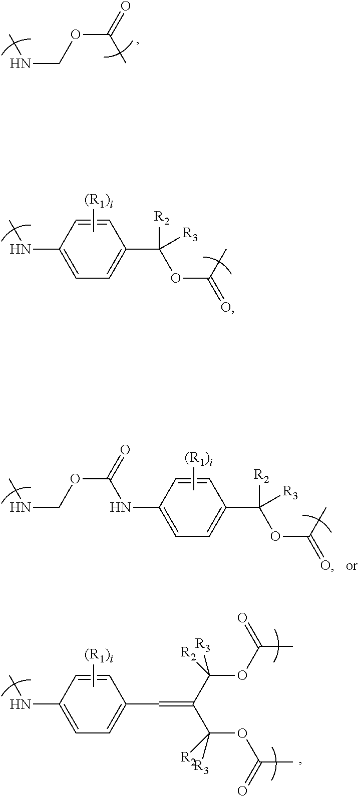

wherein each R₁, R₂ and R₃ is independently a hydrogen, a C₁-C₆ alkyl group, a halogen, a C₁-C₆ alkoxy group; and
X is a group comprising a cytotoxic moiety, and n is 1 or 2.

In yet another aspect, the invention generally relates to a birth control device. The device comprises: (1) a reservoir for holding a unit dosage comprising a contragestive and/or contraceptive effective amount of a unit dosage, the reservoir being in a shape and being made of a material suitable for implantation in a subject's uterus for a period longer than 1 month; and (2) an opening on the reservoir allowing a continuous and controlled release of the unit dosage. The unit dosage comprises a compound of Formula I

T-A₁-A₂-L-(X)ₓ (I)

or a pharmaceutically acceptable salt or ester thereof, and a pharmaceutically acceptable carrier, wherein

T is a terminal group;

each of A₁ and A₂ is independently an amino-acid group;

L is a single bond,

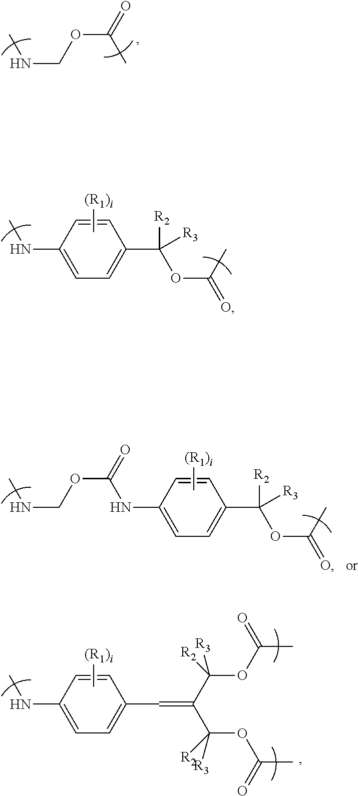

wherein each R₁, R₂ and R₃ is independently a hydrogen, a C₁-C₆ alkyl group, a halogen, a C₁-C₆ alkoxy group; and
X is a group comprising a cytotoxic moiety, and n is 1 or 2.

In yet another aspect, the invention generally relates to a birth control device. The device includes: a reservoir for holding a unit dosage comprising a contragestive and/or contraceptive effective amount of a unit dosage, the reservoir being in a shape and being made of a material suitable for implantation in a subject's uterus for a period longer than 1 month; and an opening on the reservoir allowing a continuous and controlled release of the unit dosage comprising a cytotoxic compound.

In yet another aspect, the invention generally relates to a unit dosage formulation useful as a contragestive and/or contraceptive agent. The unit dosage formulation includes a cytotoxic compound or a pharmaceutically acceptable salt or ester thereof, in an amount effective in contragestion or contraception, and a pharmaceutically acceptable carrier.

In yet another aspect, the invention generally relates to a method for birth control comprising administering to the uterus of a subject in need thereof a contragestive and/or contraceptive effective amount of a unit dosage. The unit dosage formulation includes a cytotoxic compound or a pharmaceutically acceptable salt or ester thereof, and a pharmaceutically acceptable carrier.

### BRIEF DESCRIPTION OF THE DRAWINGS

FIG. 1 shows the structure of an exemplary compound of the invention.

FIG. 2 depicts illustrative sketches of exemplary embodiments of devices according to the invention.

FIG. 3 depicts an illustrative sketch of another exemplary embodiment of the device according to the invention.

### DETAILED DESCRIPTION OF THE INVENTION

The invention provides di-peptide conjugated contraceptive agents, methods for their preparation, pharmaceutical compositions, devices and uses thereof in the prevention of pregnancy.

The invention overcomes a number of deficiencies commonly seen in conventional birth control methods including undesirable failure rates, side effects and health risks. In particular, the invention offers novel and effective birth control that can last for an extended period of time such as 6 months or longer with minimal side effects or health risks.

The cathepsins are a family of cysteine proteases characterized by the presence of a cysteine residue in the catalytic site of the enzyme. They function in the normal physiological as well as pathological degradation of connective tissue. Cysteine proteases have been associated with a number of diseases and harmful conditions (e.g., arthritis, muscular dystrophy, inflammation, tumor invasion, glomerulonephritis, malaria, periodontal disease and atherosclerosis). Cathepsins play a major role in intracellular protein degradation, turnover and remodeling. Increased levels of Cathepsin B (CB), one of several known cathepsins, and redistribution of the enzyme are found in tumors, suggesting a role for cathepsin B in tumor invasion and metastasis. (Kos, et al. 1996 Oncology Reports 5: 1349-1361.) Aberrant CB activity is also connected to rheumatoid arthritis, osteoarthritis, pneumocystis carinii, acute pancreatitis, inflammatory airway disease and bone and joint disorders. Recent studies have also suggested that CB plays a pivotal role in Alzheimer's disease and other dementing conditions. In the body, normally active CB occurs only within lysosomes and does not occur outside cells under normal conditions.

Without wishing to be bound by the theory, it is believed that after the egg is fertilized in the ovary it becomes a trophoblast, traveling down the fallopian tube (also known as oviducts or uterine tubes) for about seven days until it enters the uterus. At this point the trophoblast has divided into about eight cells, living entirely off the energy stored in its yolk. Once inside the uterus, the trophoblast must secure a new food supply, which it obtains by implanting itself into the wall of the uterus. For this purpose it begins to secrete Cathepsin B (CB) and other enzymes in order to penetrate the wall. This is the same technique and the same enzyme metastatic cancer cells use to penetrate basement membrane. Trophoblasts secrete CB in amounts sufficient for invasion of the uterine wall. (Alfonso, et al. 1997 “The expression and function of cystatin C and CB and Cathepsin L during mouse embryo implantation and placentation” Development 124, 3415-3425; Alfonso, et al. 1999 “Expression of cathepsin proteinases by mouse trophoblasts in vivo and in vitro” Developmental dynamics 216, 374-384.)

The invention takes advantage of this feature such that a CB-cleavable “prodrug” of a cytotoxic (e.g., antitumor) agent is strategically administered where the trophoblasts are expected to implant. The invention takes advantage of the characteristics of cytotoxic antitumor compounds of targeting rapidly dividing cells. A trophoblast consists 100% of rapidly dividing cells, generally 8 or 16 in number, every one of them on an external surface. In comparison, a typical small tumor of only 1 cm in diameter contains approximately 1,000,000,000 cells, relatively few of them on an external surface, and relatively few of them actively dividing at any given time. To reach an internally situated cell, the drug must penetrate quite far, which can take a long time, whereas a cell on the surface is instantaneously accessed by the agent as soon as it arrives on the scene. Thus, killing an 8- or 16-cell trophoblast is expected to be rapid and complete, requiring only enough drug for 8 or 16 cells.

CB secreted by the trophoblasts will cleave the linker (e.g., a peptide linker) masking the cytotoxic agent (e.g., ADM), releasing the agent free on the surface of the trophoblast. The result is that implantation of the trophoblast will be prevented. CB is sufficiently reactive in vivo to cause PADM to release a cytotoxic quantity of ADM. (See, Shao, et al. 2011 “Cathepsin B cleavable novel prodrug Ac-Phe-Lys-PABC-ADM enhances efficacy at reduced toxicity in treating gastric cancer peritoneal carcinomatosis” Cancer. doi: 10.1002/cncr.26596; expressly incorporated herein by reference for all purposes.) Due to the high potency of ADM, an extremely small amount is required to be effective in killing the trophoblast. It is believed to take about 1 million molecules of ADM to kill one cell; therefore, at least 8 million molecules are needed. This amount, however, correspond to 1.3×10⁻¹⁴ g, or 1.3×10⁻¹¹ mg. And 100 times of this minimum amount is 1.3×10⁻⁹ mg, an infinitesimal amount. Even when this amount is released every day for ten years, the total would be only around 5×10⁻⁶ mg, impossible to cause adverse effect on the patient.

There are several approaches for implanting an agent such as PADM into the uterus such that a million molecules/day are released over the course of a year or any other period of time. Agents such as PADM a tremendous advantage over contraceptives presently in wide use such as powerful steroids that cause numerous side effects.

The core approach of the invention in effectively preventing pregnancy is to deliver a cytotoxic agent to the trophoblast but nowhere else by adding an inactivating chemical mask to the cytotoxic agent, which mask can be removed only by CB.

A small molecule conjugate of the invention, for example a di-peptide-doxorubicin conjugate, is stable in the body because there is normally no free CB to break it down and release free drug anywhere, including organs particularly attached by cytotoxic drugs: bone marrow, GI tract and heart. When the conjugate encounters the trophoblast, free CB on its perimeter removes the masking group and releases the free doxorubicin, which readily kill or damage trophoblast cells. The conjugate molecule remains intact anywhere else in the body due to the lack of CB to break down the masking group. The conjugate is known to be stable in human blood. (Dubowchik, et al. 1998 Bioorg Med Chem Lett. 8: 3341-3346; Dubowchik, et al. 1998 Bioorg Med Chem Lett. 8: 3347-3352; 20.)

In one aspect, the invention generally relates to a unit dosage formulation useful as a contragestive and/or contraceptive agent, comprising a compound of Formula I

T-A₁-A₂-L-(X)ₙ (I)

or a pharmaceutically acceptable salt or ester thereof, in an amount effective in contragestion or contraception, and a pharmaceutically acceptable carrier, wherein

T is a terminal group;

each of A₁ and A₂ is independently an amino-acid group;

L is a single bond,

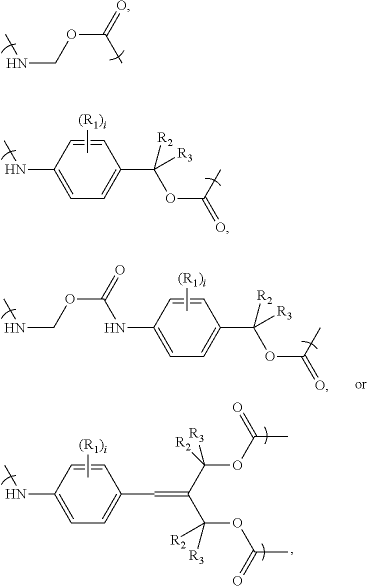

wherein each R₁, R₂ and R₃ is independently a hydrogen, a C₁-C₆ alkyl group, a halogen, a C₁-C₆ alkoxy group; and
X is a group comprising a cytotoxic moiety, and n is 1 or 2.

In certain preferred embodiments, A₂ is Lys and A₁ is Phe or Val with A₁ preferably being Phe.

In certain embodiments, X is

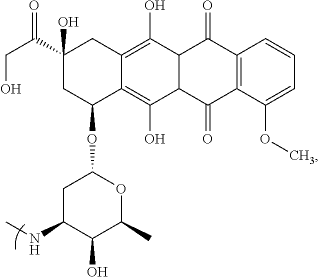 [SMILES: CN[C@H]1C[C@H](O[C@H]2C[C@](O)(C(=O)CO)CC3=C(O)C4C(=O)c5cccc(OC)c5C(=O)C4C(O)=C32)O[C@@H](C)[C@H]1O]

### and L is

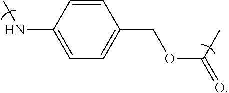

And T, for example, may be selected from R—(C═O)— (wherein R is a C₁-C₆ alkyl), D-amino acid groups, trimethylated D-amino acid cations. For example, T may be D-Phe or trimethylated D-Phe.

In certain preferred embodiments, T is selected from t-butyloxycarbonyl (BOC), benzoyl, and phenylacetyl.

In certain preferred embodiments, T is selected a carbobenzoxy group having the formula:

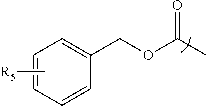

wherein R₅ is one or more of a C₁-C₆ alkyl, a C₁-C₆ alkoxy group, halogen, —CN, methylsulfinyl, carbomethoxy, carboxy, dimethylamino, trimethlyammonio, and (m,p-CH₂OCH₂—).

Each R₁, R₂ and R₃ is independently a hydrogen, a C₁-C₆ alkyl group (e.g., methyl, ethyl, propyl, isopropyl, n-butyl, isobutyl), a halogen (e.g., F, Cl, Br, I), a C₁-C₆ alkoxy group (e.g., methoxy, ethyoxy, propoxy).

X may be a cytotoxic moiety derived from a compound selected from Table 2. In certain embodiments, X is a moiety derived from a compound selected from anthracyclines, actinomycins, mitomycins, bleomycins, plicamycins. In certain embodiments, X is a moiety derived from a cytotoxic compound selected Table 3.

TABLE 2
Cytotoxic Agents

| Alkylators | DNA intercalators | Nucleotide mimics |
|---|---|---|
| melphalan (L-PAM) | doxorubicin (adriamycin) | 5-FU (fluorouracil) |
| BCNU (Carmustine) | daunomycin | 6-mercaptopurine |
| CCNU (lomustine) | dapdox | 5-azacytidine |
| Me-CCNU | methoxymorpholino dox | 6-azauridine |
| Chlorambucil | 2-pyrrolamido dox | cytosine arabinoside |
| Mechlorethiminebusulfan | | 6-thioguanine ara C |
| Cytoxan | | |
| mitomycin C | | |
| neocarzinostatin | | |
| Antibiotics | Angiogenesis inhibitor | Folate mimics |
| streptozotocin | 17-amino-geldanamycin | methotrexate |
| dactinomycin | | |
| streptonigrin | | |
| Differentiation inducers | Fatty acids | |
| SAHA (suberoamido | bromopyruvic acid | |
| anilide hydroxamic acid) | dichloroacetic acid | |
| Butyrate | 13-methyltetradecanoic | |
| phenylbutyrate | acid | |
| OSU-HDAC2 | | |
| Alkaloids | Mitosis interference | Others |
| actinomycin D | camptothecin | trimetrexate |
| vincristine | topotecan | carboplatin |
| vinblastine | CPT-11 | cis-platin |
| amsacrine | paclitaxel (taxol) | tetraplatin |
| gemcitabine | docetaxel | thio TEPA |
| bleomycin (tallysomycin) | BES (bisethylspermine) | etoposide (VP-16) |
| maytansine | difluoromethylornithine | teniposide (VM-26) |
| mithramycin | | colchicine |
| | | tamoxifen |
| | | hydroxyurea |
| | | sulforaphane |

In certain preferred embodiments, X is a moiety derived from doxorubicin, methotrexate (MTX), melphalan, mitomycin C, suberoylanilide hydroxamic acid (SAHA), fluorouracil (5-FU), camptothecin, paclitaxel, docetaxel, vincristine, bleomycin, tallysomycin, etoposide, and bisethylspermine, and derivatives and analogs thereof. Structures of exemplary agents are provided in Table 3.

TABLE 3
Structures of Select Cytotoxic Agents

| | |
|---|---|
| 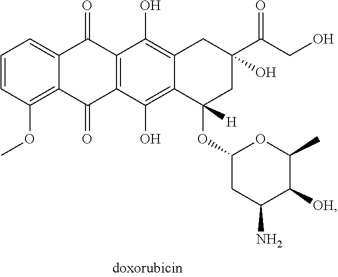 [SMILES: COc1cccc2c1C(=O)c1c(O)c3c(c(O)c1C2=O)C[C@@](O)(C(=O)CO)C[C@@H]3O[C@H]1C[C@H](N)[C@H](O)[C@H](C)O1] | 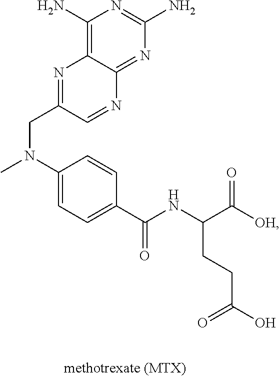 [SMILES: CN(Cc1cnc2nc(N)nc(N)c2n1)c1ccc(C(=O)NC(CCC(=O)O)C(=O)O)cc1] |
| 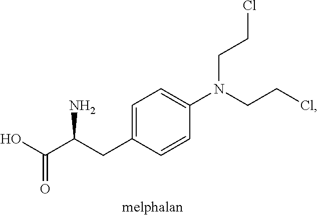 [SMILES: N[C@@H](Cc1ccc(N(CCCl)CCCl)cc1)C(=O)O] | 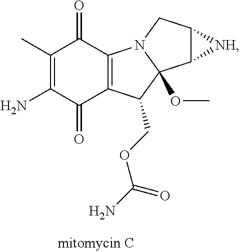 [SMILES: CO[C@@]12[C@H](COC(N)=O)C3=C(C(=O)C(C)=C(N)C3=O)N1C[C@@H]1N[C@@H]12] |
| 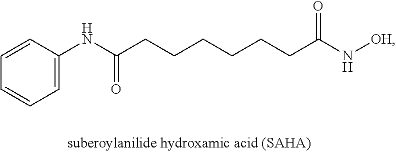 [SMILES: O=C(CCCCCCC(=O)Nc1ccccc1)NO] | 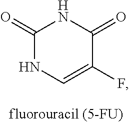 |
| 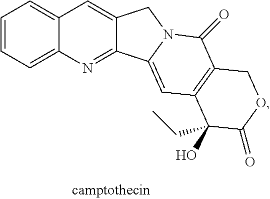 [SMILES: CC[C@@]1(O)C(=O)OCc2c1cc1n(c2=O)Cc2cc3ccccc3nc2-1] | 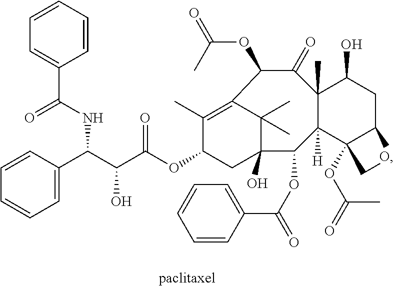 [SMILES: CC(=O)O[C@H]1C(=O)[C@@]2(C)[C@H]([C@H](OC(=O)c3ccccc3)[C@]3(O)C[C@H](OC(=O)[C@H](O)[C@@H](NC(=O)c4ccccc4)c4ccccc4)C(C)=C1C3(C)C)[C@]1(OC(C)=O)CO[C@@H]1C[C@@H]2O] |
| 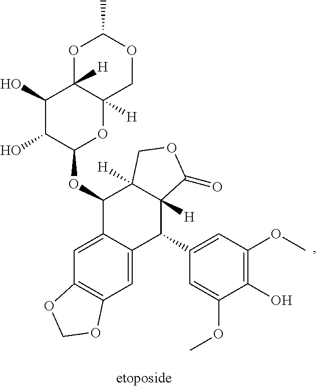 | 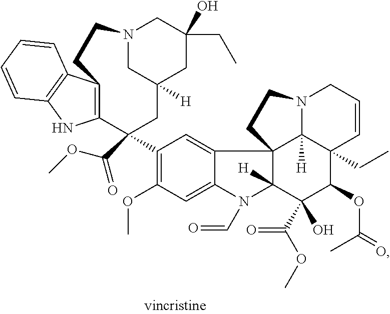 |

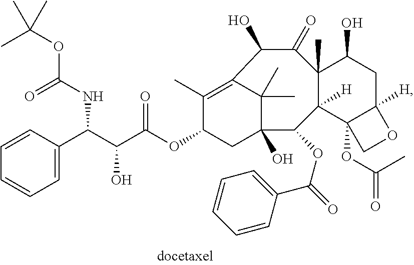

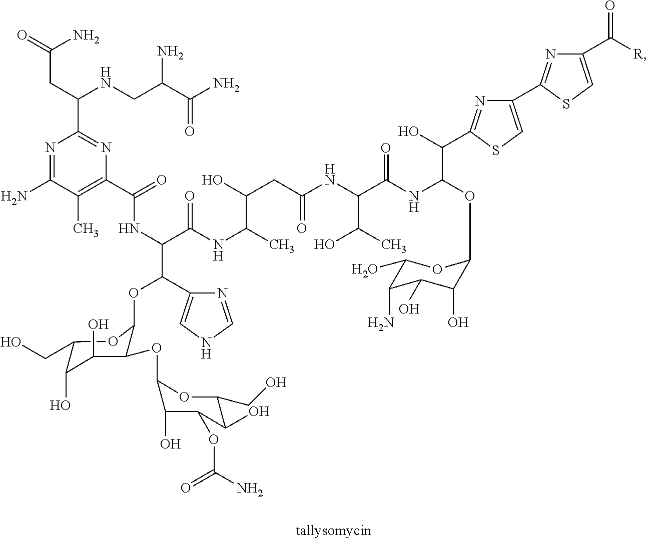

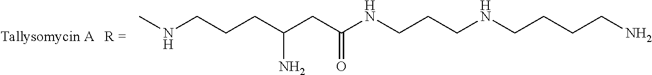 [SMILES: CNCCCC(N)CC(=O)NCCCNCCCCN]

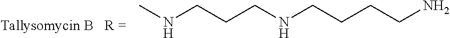

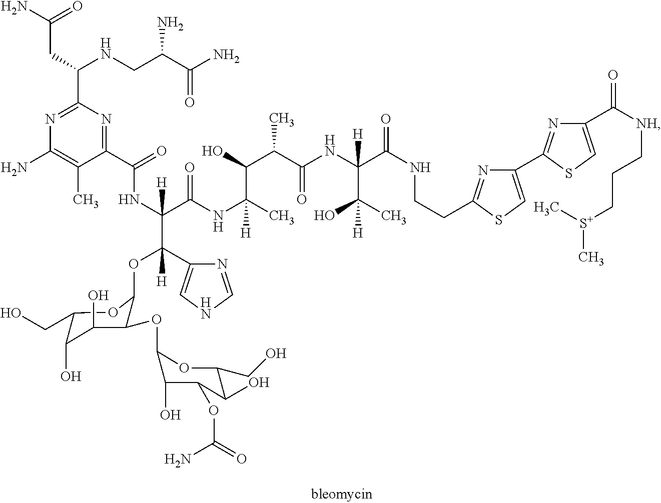 [SMILES: Cc1c(N)nc([C@H](CC(N)=O)NC[C@H](N)C(N)=O)nc1C(=O)N[C@H](C(=O)N[C@H](C)[C@@H](O)[C@H](C)C(=O)N[C@H](C(=O)NCCc1nc(-c2nc(C(=O)NCCC[S+](C)C)cs2)cs1)[C@@H](C)O)[C@@H](OC1OC(CO)C(O)C(O)C1OC1OC(CO)C(O)C(OC(N)=O)C1O)c1c[nH]cn1]

In certain embodiments, n is 1 and L is

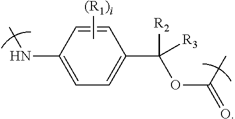

In certain preferred embodiments, n is 1 and L is

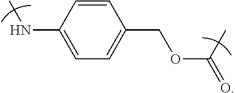

In certain embodiments, n is 1 and L is

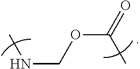

In certain embodiments, n is 1 and L is a single bond.

In certain embodiments, n is 2 and L is

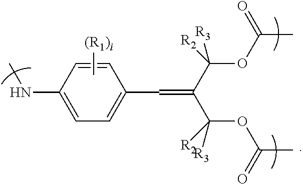

The unit dosage formulation may include from about 1.0 μg to about 100 mg of a cytotoxic agent (e.g., from about 2.0 μg to about 100 mg, from about 5.0 μg to about 100 mg, from about 10.0 μg to about 100 mg, from about 50 μg to about 100 mg, from about 100 μg to about 100 mg, from about 500 μg to about 100 mg, from about 1.0 mg to about 100 mg, from about 5.0 mg to about 100 mg, from about 10 mg to about 100 mg, from about 50 mg to about 100 mg, from about 1.0 μg to about 50 mg, from about 1.0 μg to about 10 mg, from about 1.0 μg to about 5 mg, from about 1.0 μg to about 1.0 mg). The unit dosage formulation may be sufficient to be effective in preventing pregnancy for a period of 1 month or longer (e.g., 1 month or longer, 3 months or longer, 6 months or longer, 12 months or longer). It is noted that fertility can be restored whenever desired, simply by removing the device of the invention from the uterus.

In another aspect, the invention generally relates to a method for birth control comprising administering to the uterus of a subject in need thereof a contragestive and/or contraceptive effective amount of a unit dosage comprising a compound of Formula I

T-A₁-A₂-L-(X)ₙ (I)

or a pharmaceutically acceptable salt or ester thereof, and a pharmaceutically acceptable carrier, wherein

T is a terminal group;

each of A₁ and A₂ is independently an amino-acid group;

L is a single bond,

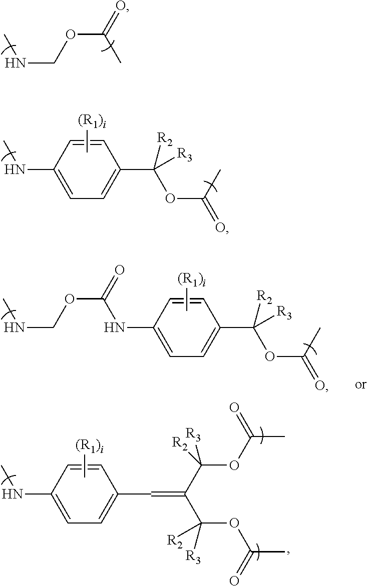

wherein each R₁, R₂ and R₃ is independently a hydrogen, a C₁-C₆ alkyl group, a halogen, a C₁-C₆ alkoxy group; and
X is a group comprising a cytotoxic moiety, and n is 1 or 2.

In yet another aspect, the invention generally relates to a birth control device. The device comprises: (1) a reservoir for holding a unit dosage comprising a contragestive and/or contraceptive effective amount of a unit dosage, the reservoir being in a shape and being made of a material suitable for implantation in a subject's uterus for a period longer than 1 month; and (2) an opening on the reservoir allowing a continuous and controlled release of the unit dosage. The unit dosage comprises a compound of Formula I

T-A₁-A₂-L-(X)ₙ (I)

or a pharmaceutically acceptable salt or ester thereof, and a pharmaceutically acceptable carrier, wherein

T is a terminal group;

each of A₁ and A₂ is independently an amino-acid group;

L is a single bond,

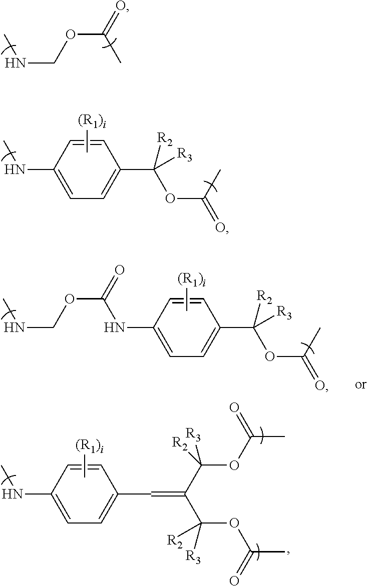

wherein each R₁, R₂ and R₃ is independently a hydrogen, a C₁-C₆ alkyl group, a halogen, a C₁-C₆ alkoxy group; and
X is a group comprising a cytotoxic moiety, and n is 1 or 2.

In yet another aspect, the invention generally relates to a birth control device. The device includes: a reservoir for holding a unit dosage comprising a contragestive and/or contraceptive effective amount of a unit dosage, the reservoir being in a shape and being made of a material suitable for implantation in a subject's uterus for a period longer than 1 month; and an opening on the reservoir allowing a continuous and controlled release of the unit dosage comprising a cytotoxic compound.

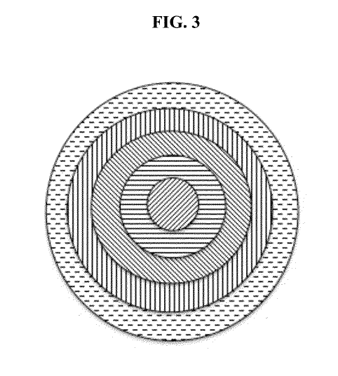

The device may take any suitable size or shape, e.g., a closed cylindrical tube with the active agent stored therein and with one or more openings either at one or both ends of the tube and/or on the side wall of the tube, as schematically illustrated in FIG. 2. The opening is designed to allow a controlled amount of the agent to diffuse into the surroundings over an extended period of time. The opening may be a membrane-type or a porous orifice, for example. FIG. 3 depicts another exemplary embodiment of the device according to the invention, where an agent is infused in a spherical, pill-like implant (e.g., a biodegradable polymer matrix). The drug-infused spherical pill is designed to dissolve very slowly. The spherical pill is made in layers (four shown in FIG. 3 but can have more than 4 layers when needed). Each added layer has a lower concentration of drug so that the average drug delivery rate stays relatively constant throughout the process.

In yet another aspect, the invention generally relates to a unit dosage formulation useful as a contragestive and/or contraceptive agent. The unit dosage formulation includes a cytotoxic compound or a pharmaceutically acceptable salt or ester thereof, in an amount effective in contragestion or contraception, and a pharmaceutically acceptable carrier.

In yet another aspect, the invention generally relates to a method for birth control comprising administering to the uterus of a subject in need thereof a contragestive and/or contraceptive effective amount of a unit dosage. The unit dosage formulation includes a cytotoxic compound or a pharmaceutically acceptable salt or ester thereof, and a pharmaceutically acceptable carrier.

The unit dosage may include from about 0.1 μg to about 50 mg of the cytotoxic compound (e.g., from about 0.2 μg to about 50 mg, from about 0.5 μg to about 50 mg, from about 1.0 μg to about 50 mg, from about 0.1 μg to about 20 mg, from about 0.1 μg to about 10 mg) and is sufficient to provide effective birth control for a period of 1 month or longer (e.g., 1 month or longer, 3 months or longer, 6 months or longer, 12 months or longer).

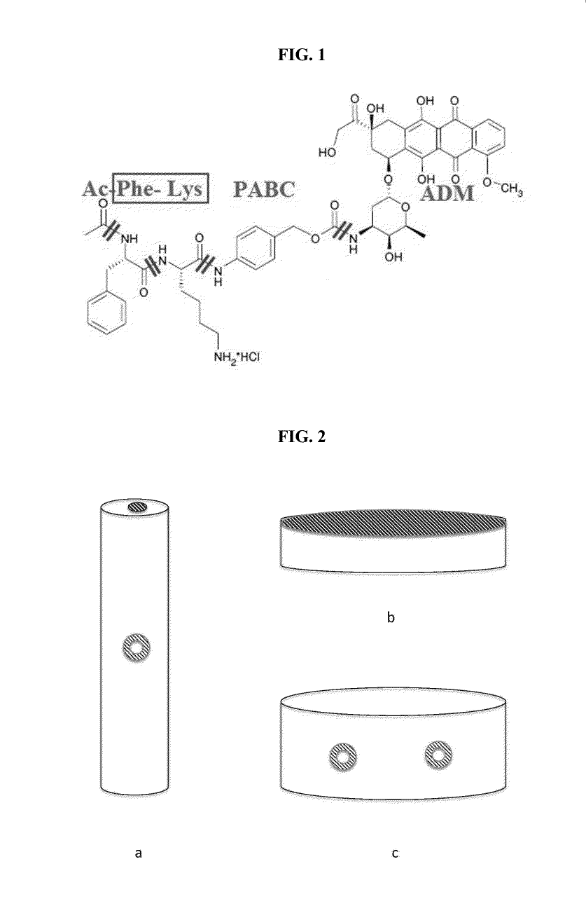

An exemplary compound of the invention, as shown in FIG. 1, is Ac-Phe-Lys-PABC-ADM (PADM) (hydrochloride), which is a conjugate of a dipeptide and ADM with the linkage, para-aminobenzyloxycarbonyl (PABC), a self-immolative spacer. (Dubowchik, et al. 1998 Bioorg Med Chem Lett. 8: 3341-3346; Dubowchik, et al. 1998 Bioorg Med Chem Lett. 8: 3347-3352; 20. Dubowchik, et al. 2002 Bioconjug Chem. 13: 855-869.) The agent is inactive when there is little activated CB, such as normal tissues and peripheral blood, thus avoiding the side effects on normal tissue. Activated CB cleaves the Phe-Lys dipeptide at the Lys-PABC bond. Then, the exposed PABC spacer spontaneously decarboxylates upon deacylation and free ADM molecules are released. The pure in vitro release study of PADM showed that the half-life of ADM release at 37° C. was 16 min in CB solution, but no changes were observed over 6-7 h in human plasma.

PADM was synthesized according to previously reported chemical process. (Dubowchik, et al. 1998 Bioorg Med Chem Lett. 8: 3341-3346; Dubowchik, et al. 1998 Bioorg Med Chem Lett. 8: 3347-3352; 20. Dubowchik, et al. 2002 Bioconjug Chem. 13: 855-869.) The molecular weight of PADM hydrochloride is 1045.50. In terms of equivalent mole content, 1.8 mg PADM hydrochloride is equivalent to 1 mg ADM hydrochloride (molecular weight 579.99). Other agents were obtained commercially, including Doxorubicin Hydrochloride for Injection (ADM) (Pharmacia, Milan, Italy) 10 mg/vial, RPMI-1640 medium (HyClone, NZ, USA) and Standard Newborn Bovine Serum (Zhengzhou Ben BioTech Co., Ltd., Zhengzhou, China) for cell culture, Propidine Iodide (PI) agents kit (Beckman coulter, CA, USA) for flow cytometric analysis, and rabbit anti-Cathepsin B polyclonal antibody (Lot No. 3190-100, BioVision, CA, USA) and peroxidase-conjugated Affinipure goat anti-rabbit IgG(H+L) (Lot No. 88813, Jackson ImmunoResearch, PA, USA) for immunohistochemical study.

It is noted that the invention also includes compositions and methods for emergency contraception (e.g., use of PADM) up to seven days after unprotected intercourse. As it takes about 7 days for the fertilized egg to travel down the fallopian tube to the uterus. Most available contraceptives don't allow have this option. Mifepristone, for example, is also used as a post-coital contraceptive, but one have to use a full dose, with risk of side effects, whereas with PADM and one need only a very small dose, much too small to create side effects, thereby making it an advantageous method in an emergency.

In this specification and the appended claims, the singular forms “a,” “an,” and “the” include plural reference, unless the context clearly dictates otherwise.

Unless defined otherwise, all technical and scientific terms used herein have the same meaning as commonly understood by one of ordinary skill in the art. Although any methods and materials similar or equivalent to those described herein can also be used in the practice or testing of the present disclosure, the preferred methods and materials are now described. Methods recited herein may be carried out in any order that is logically possible, in addition to a particular order disclosed.

### INCORPORATION BY REFERENCE

References and citations to other documents, such as patents, patent applications, patent publications, journals, books, papers, web contents, have been made in this disclosure. All such documents are hereby incorporated herein by reference in their entirety for all purposes. Any material, or portion thereof, that is said to be incorporated by reference herein, but which conflicts with existing definitions, statements, or other disclosure material explicitly set forth herein is only incorporated to the extent that no conflict arises between that incorporated material and the present disclosure material. In the event of a conflict, the conflict is to be resolved in favor of the present disclosure as the preferred disclosure.

### EQUIVALENTS

The representative examples are intended to help illustrate the invention, and are not intended to, nor should they be construed to, limit the scope of the invention. Indeed, various modifications of the invention and many further embodiments thereof, in addition to those shown and described herein, will become apparent to those skilled in the art from the full contents of this document, including the examples and the references to the scientific and patent literature included herein. The examples contain important additional information, exemplification and guidance that can be adapted to the practice of this invention in its various embodiments and equivalents thereof.

What is claimed is:

1. A unit dosage formulation useful as a contragestive and/or contraceptive agent, comprising a compound of Formula I
T-A₁-A₂-L-(X)ₙ (I)
or a pharmaceutically acceptable salt or ester thereof, in an amount effective in contragestion or contraception, and a pharmaceutically acceptable carrier, wherein
T is a terminal group;
each of A₁ and A₂ is independently an amino-acid group;
L is a single bond, 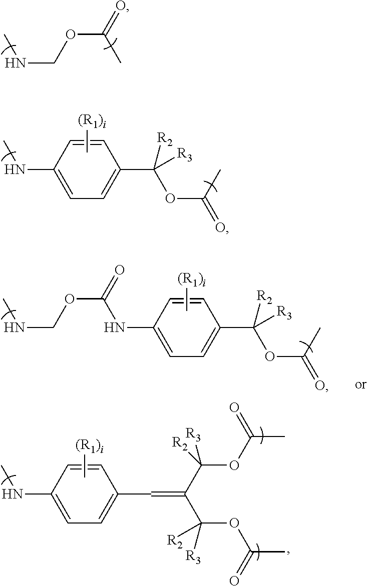
wherein each R₁, R₂ and R₃ is independently a hydrogen, a C₁-C₆ alkyl group, a halogen, a C₁-C₆ alkoxy group; and
X is a group comprising a cytotoxic moiety, and n is 1 or 2.

2. The unit dosage formulation of claim 1, wherein n is 1 and L is 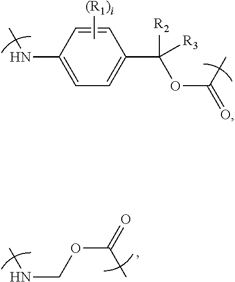
or a single bond.

3. The unit dosage formulation of claim 1, wherein n is 2 and L is

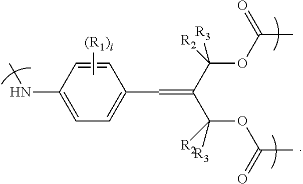

4. The unit dosage formulation of claim 2, wherein n is 1 and L is

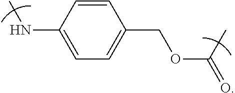

5. The unit dosage formulation of claim 1, wherein A₂ is Lys and A₁ is Phe or Val.

6. (canceled)

7. The unit dosage formulation of claim 5, wherein X is 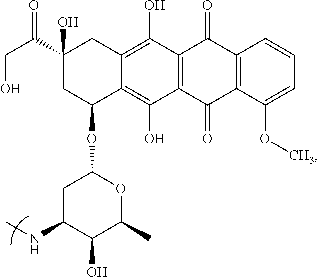 [SMILES: CN[C@H]1C[C@H](O[C@H]2C[C@](O)(C(=O)CO)Cc3c(O)c4c(c(O)c32)C(=O)c2c(OC)cccc2C4=O)O[C@@H](C)[C@H]1O]
and L is

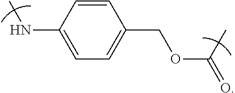

8. The unit dosage formulation of claim 1, wherein T is selected from R—(C═O)— (wherein R is a C₁-C₆ alkyl), D-amino acid groups, and trimethylated D-amino acid cations.

9. (canceled)

10. (canceled)

11. The unit dosage formulation of claim 1, wherein X is a moiety derived from a cytotixic compound selected from anthracyclines, actinomycins, mitomycins, bleomycins, plicamycins.

12. The unit dosage formulation of claim 1, wherein X is a moiety derived from a cytotoxic compound selected from Table 2.

13. (canceled)

14. The unit dosage formulation of claim 1, comprising from about 1.0 μg to about 100 mg of the agent and is sufficient to be effective for a period of 1 month or longer.

15. A method for birth control comprising administering to the uterus of a subject in need thereof a contragestive and/or contraceptive effective amount of a unit dosage comprising a compound of Formula I
T-A₁-A₂-L-(X)ₙ (I)
or a pharmaceutically acceptable salt or ester thereof, and a pharmaceutically acceptable carrier, wherein
T is a terminal group;
each of A₁ and A₂ is independently an amino-acid group;
L is a single bond, 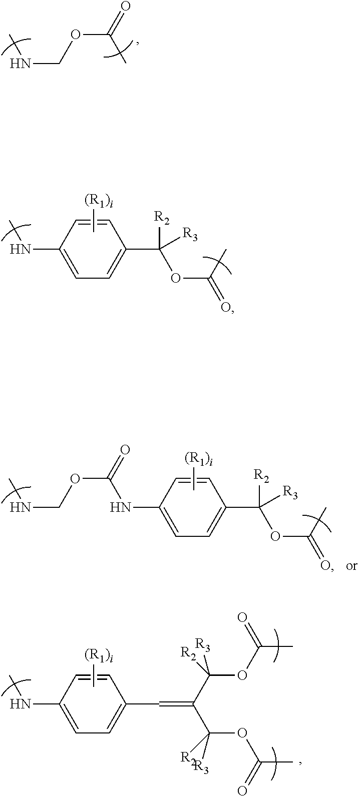
wherein each R₁, R₂ and R₃ is independently a hydrogen, a C₁-C₆ alkyl group, a halogen, a C₁-C₆ alkoxy group; and
X is a group comprising a cytotoxic moiety, and n is 1 or 2.

16. The method of claim 15, wherein n is 1 and L is 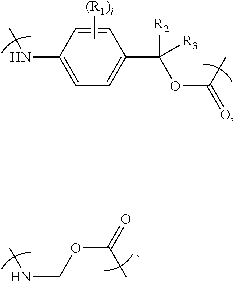
or a single bond.

17. The method of claim 15, wherein n is 2 and L is

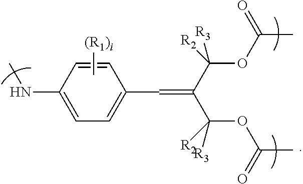

18. The method of claim 16, wherein n is 1 and L is

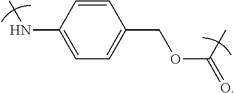

19. The method of claim 15, wherein A₂ is Lys and A₁ is Phe or Val.

20. (canceled)

21. The method of claim 20, wherein X is 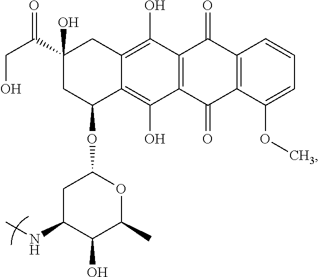 [SMILES: CN[C@H]1C[C@H](O[C@H]2C[C@](O)(C(=O)CO)Cc3c(O)c4c(c(O)c32)C(=O)c2c(OC)cccc2C4=O)O[C@@H](C)[C@H]1O]
and L is

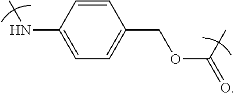

22. The method of claim 15, wherein T is selected from R—(C═O)— (wherein R is a C₁-C₆ alkyl), D-amino acid groups, trimethylated D-amino acid cations.

23. (canceled)

24. (canceled)

25. The method of claim 15, wherein X is a cytotoxic-active moiety derived from a cytotoxic compound selected from anthracyclines, actinomycins, mitomycins, bleomycins, plicamycins.

26. The method of claim 15, wherein X is a moiety derived from a cytotoxic compound selected from Table 2.

27. (canceled)

28. The method of claim 15, wherein the unit dosage comprises from about 1.0 μg to about 100 mg of the agent and the method is sufficient to provide effective birth control for a period of 1 month or longer.

29. A birth control device comprising:
a reservoir for holding a unit dosage comprising a contragestive and/or contraceptive effective amount of a unit dosage, the reservoir being in a shape and being made of a material suitable for implantation in a subject's uterus for a period longer than 1 month; and
an opening on the reservoir allowing a continuous and controlled release of the unit dosage comprising a compound of Formula I
T-A₁-A₂-L-(X)ₙ (I)
or a pharmaceutically acceptable salt or ester thereof, and a pharmaceutically acceptable carrier, wherein
T is a terminal group;
each of A₁ and A₂ is independently an amino-acid group;
L is a single bond, 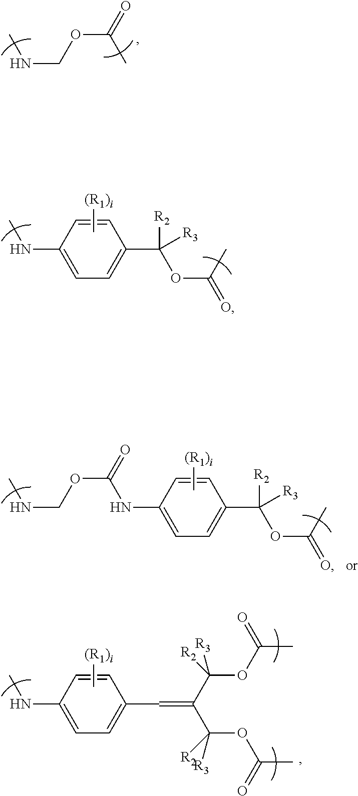
wherein each R₁, R₂ and R₃ is independently a hydrogen, a C₁-C₆ alkyl group, a halogen, a C₁-C₆ alkoxy group; and
X is a group comprising a cytotoxic moiety, and n is 1 or 2.

30-34. (canceled)

35. The device of claim 34, wherein X is 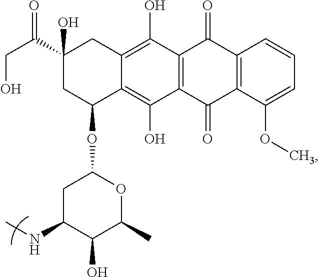 [SMILES: CN[C@H]1C[C@H](O[C@H]2C[C@](O)(C(=O)CO)Cc3c(O)c4c(c(O)c32)C(=O)c2c(OC)cccc2C4=O)O[C@@H](C)[C@H]1O]
and L is

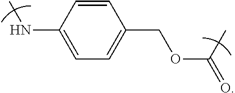

36. The device of claim 29, wherein T is selected from R—(C═O)— (wherein R is a C₁-C₆ alkyl), D-amino acid groups, trimethylated D-amino acid cations.

37-39. (canceled)

40. The device of claim 29, wherein X is a moiety derived from a cytotoxic compound selected from Table 2.

41. (canceled)

42. The device of claim 29, wherein the unit dosage comprises from about 1.0 μg to about 100 mg of the agent and the method is sufficient to provide effective birth control for a period of 1 month or longer.

43-46. (canceled)

47. A unit dosage formulation useful as a contragestive and/or contraceptive agent, comprising a cytotoxic compound or a pharmaceutically acceptable salt or ester thereof, in an amount effective in contragestion or contraception, and a pharmaceutically acceptable carrier.

48-56. (canceled)
